(19) United States
(12) Patent Application Publication
(10) Pub. No.: US 2026/0007389 A1
(43) Pub. Date: Jan. 8, 2026

(54) SYSTEMS AND METHODS OF 3D VOLUMETRIC ULTRASOUND IMAGING

(71) Applicant: University of Pittsburgh - Of the Commonwealth System of Higher Education, Pittsburgh, PA (US)

(72) Inventors: Kang Kim, Pittsburgh, PA (US); Zhiyu Sheng, Pittsburgh, PA (US)

(73) Assignee: University of Pittsburgh - Of the Commonwealth System of Higher Education, Pittsburgh, PA (US)

(21) Appl. No.: 18/992,165

(22) PCT Filed: Jun. 30, 2023
(86) PCT No.: PCT/US2023/026772

§ 371 Date: Jan. 7, 2025

Related U.S. Application Data
(60) Provisional application No. 63/359,415, filed on Jul. 8, 2022.

(51) Int. Cl. A61B 8/00

(52) U.S. Cl. CPC A61B 8/4488; A61B 8/5207

(57) ABSTRACT

A sparse-array ultrasound imaging system includes an ultrasound transducer with a transducer array having a plurality of transducer elements defining an element layer. One or more lens layers are positioned over the element layer, and the one or more lens layers are configured to provide a time delay to at least some of the signals emitted by at least some of the transducer elements.

### CROSS REFERENCE TO RELATED APPLICATIONS

This claims the benefit of U.S. Provisional Application No. 63/359,415, filed Jul. 8, 2022, which is incorporated herein by reference.

### FIELD

The present disclosure relates to apparatus and methods of ultrasound imaging, and, in particular, to 3D volumetric ultrasound imaging.

### BACKGROUND

Ultrasound is an important imaging modality, especially in biomedical applications, for its non-invasiveness, real-time capability, compactness, and affordability. Conventional ultrasound imaging probes provide 2-dimensional (2D) slice images of a target area, such as an organ or other internal area of a body. These 2D slice images can leads to inaccurate measurements and/or biased interpretation of data, because the analysis must perform under 2D geometric assumptions, which can never be perfectly satisfied since the target area is present in 3D space. This problem can be further compounded for moving target areas, such as the heart.

In clinical applications, inaccuracies and misinterpretations can lead to misdiagnosis because the Accordingly, there is a need for improved ultrasound imaging techniques.

### SUMMARY

In some embodiments, a sparse-array ultrasound imaging system is provided. The system includes an ultrasound transducer comprising a transducer array having a plurality of transducer elements defining an element layer, the plurality of transducer elements being configured to emit ultrasound signals. The system also includes one or more lens layers positioned over the element layer, with the one or more lens layers comprising a plurality of lens elements that correspond, respectively, with the plurality of transducer elements. The one or more lens layers are configured to affect a time delay of at least some of the signals emitted by the transducer elements.

In some embodiments, the one or more lens layers comprise a plurality of groups, and lens elements within each group of the plurality of groups are of the same time. The plurality of groups include at least a first group and a second group, and the one or more lens layers in the first group provide a first time delay of ultrasound signals emitted by corresponding ones of transducer elements, and the one or more lens layers in the second group provide a second time delay to ultrasound signals emitted by corresponding ones of transducer elements, and the first time delay is different from the second time delay.

The one or more lens layers can be configured to be coupled to the ultrasound transducer of the sparse-array ultrasound imaging system. In some embodiments, the one or more lens layers can be coupled to the ultrasound transducer at a first spacing relative to the transducer array, and the first spacing is adjustable.

In some embodiments, a number of receive elements of the transducer array is reduced by a channel reduction factor and wherein the channel reduction factor is 8/5 or greater, 3 or greater, 4 or greater, 6 or greater, or 8 greater. In some embodiments where the channel reduction factor is 8/5 or greater, 3 or greater, 4 or greater, 6 or greater, or 8 greater, the channel reduction factor can also be less than 16, or less than 12.

In some embodiments, a number of transmit elements of the transducer array is reduced by the same channel reduction factor as the number of receive elements.

In other embodiments, a method for ultrasound imaging using a sparse-array ultrasound imaging system comprises providing an ultrasound transducer comprising a transducer array having a plurality of transducer elements defining an element layer, the plurality of transducer elements configured to emit ultrasound signals; positioning one or more lens layers over the element layer, the one or more lens layers comprising a plurality of lens elements that correspond, respectively, with the plurality of transducer elements, the one or more lens layers being configured to affect a time delay of at least some of the signals emitted by the transducer elements; emitting ultrasound signals from the transducer elements; receiving reflected ultrasound signals from a target area by a plurality of active receive elements of the transducer array; combining the received reflected ultrasound signals affected by the time delay from the one or more lens layers; processing the combined signals to generate an ultrasound image of the target area. A number of the active receive elements of the transducer array can be reduced by a channel reduction factor of 8/5 or greater, 3 or greater, 4 or greater, 6 or greater, or 8 or greater.

In some embodiments where the channel reduction factor is 8/5 or greater, 3 or greater, 4 or greater, 6 or greater, or 8 greater, the channel reduction factor can also be less than 16, or less than 12.

In some embodiments, a number of active transmit elements of the transducer array is reduced by the same channel reduction factor as the number of active receive elements. The one or more lens layers can comprise a plurality of groups, and lens elements within each group of the plurality of groups are of the same type. Different lens layers with the different ones of the plurality of groups can have different lens thicknesses.

In some embodiments, the plurality of groups include at least a first group and a second group, and the one or more lens layers in the first group provide a first time delay of ultrasound signals emitted by corresponding ones of transducer elements, and the one or more lens layers in the second group provide a second time delay to ultrasound signals emitted by corresponding ones of transducer elements, and the first time delay is different from the second time delay.

In some embodiments, the one or more lens layers are configured to be coupled to the ultrasound transducer of the sparse-array ultrasound imaging system. The one or more lens layers can be coupled to the ultrasound transducer at a first spacing relative to the transducer array, and the first spacing is adjustable.

The foregoing and other objects, features, and advantages of the invention will become more apparent from the following detailed description, which proceeds with reference to the accompanying figures.

### BRIEF DESCRIPTION OF THE DRAWINGS

FIG. 1 illustrates a schematic view of an exemplary sparse-array ultrasound system.

FIG. 2 illustrates a comparison of a full-channel B-mode image with a ¼ channel AI reconstruction of a sparse-array ultrasound system.

FIG. 3 illustrates a simulation of a sparse array in various configurations.

### DETAILED DESCRIPTION

The following disclosure is presented in the context of representative embodiments that are not to be construed as being limiting in any way. This disclosure is directed toward all novel and non-obvious features and aspects of the various disclosed embodiments, alone and in various combinations and sub-combinations with one another. The disclosed methods, apparatus, and systems are not limited to any specific aspect or feature or combination thereof, nor do the disclosed embodiments require that any one or more specific advantages be present or problems be solved.

Although the operations of the disclosed methods are described in a particular, sequential order for convenient presentation, it should be understood that this manner of description encompasses rearrangement of the operations, unless a particular ordering is required by specific language set forth below. For example, operations described sequentially may in some cases be rearranged or performed concurrently. Moreover, for the sake of simplicity, the attached figures may not show the various ways in which the disclosed systems, methods, and apparatus can be used in conjunction with other things and methods.

This disclosure sometimes uses terms like “produce,” “generate,” “select,” “receive,” “exhibit,” and “provide” to describe the disclosed methods. These terms are high-level abstractions of the actual operations that are performed. The actual operations that correspond to these terms may vary depending on the particular implementation and are readily discernible by one of ordinary skill in the art.

The singular forms “a,” “an,” and “the” include the plural forms unless the context clearly dictates otherwise. The term “includes” means “comprises.” Unless the context dictates otherwise, the term “coupled” means mechanically, electrically, or electromagnetically connected or linked and includes both direct connections or direct links and indirect connections or indirect links through one or more intermediate elements not affecting the intended operation of the described system. The term “or” refers to a single element of stated alternative elements or a combination of two or more elements, unless the context clearly indicates otherwise.

Certain terms may be used such as “up,” “down,” “upper,” “lower,” and the like. These terms are used, where applicable, to provide some clarity of description when dealing with relative relationships. But, these terms are not intended to imply absolute relationships, positions, and/or orientations.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

Unless otherwise indicated, all numbers expressing quantities of components, percentages, temperatures, times, and so forth, as used in the specification or claims are to be understood as being modified by the term “about” or “approximately.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited.

As used herein, the terms “ultrasound transducer” and “transducer” have their ordinary meanings as understood by those skilled in the art of ultrasound imaging technologies. Any suitable transducer may be used. For example, in some embodiments, an ultrasound transducer may comprise a piezoelectric device. The transducers described herein are configured in arrays of multiple individual transducer elements. As used herein, the terms “transducer array” or “array” refers to a collection of transducer elements attached to a common support structure.

As used herein, the terms “transmit element” and “receive element” have their ordinary meanings as understood by those skilled in the art of ultrasound imaging technologies. For example, the term “transmit element” refers without limitation to an ultrasound transducer element which at least momentarily performs a transmit function in which an electrical signal is converted into an ultrasound signal. Similarly, the term “receive element” refers without limitation to an ultrasound transducer element which at least momentarily performs a receive function in which an ultrasound signal impinging on the element is converted into an electrical signal.

A major technical barrier that prevents the realization of 3D ultrasound in conventional ultrasound imaging systems is the extensive number of electrical channels required by a 2D full-element array probe. Given the number of electrical channels of the system, the field of view or/and image quality must be significantly compromised. The systems and methods disclosed herein, utilize a novel sensor array probe and related systems that greatly reduce the required number of electrical channels while maintaining a comparable image quality, so that a real-time, free-hand scanned, large field-of-view high-frame-rate 3D volumetric imaging can be achieved.

In some embodiments, the system and methods provide front-end beamforming through a combination of mechanical delay introduced by novel acoustic lenses and electrical delay circuits on the chosen sub-sampled elements. Artificial intelligence (AI)-based image formation technology can facilitate the beamforming challenge by reducing the number of receiving sensor elements.

In some embodiments, the mechanical delays can be tailored for specific imaging applications by flexibly/optimally adjusting the height of each element in the acoustic lens, relative to a spacing with the transducer array. The sub-sampling can also be optimized for specific applications by a flexible receive selection. The spacing can be adjusted by any suitable mechanical adjustment mechanism.

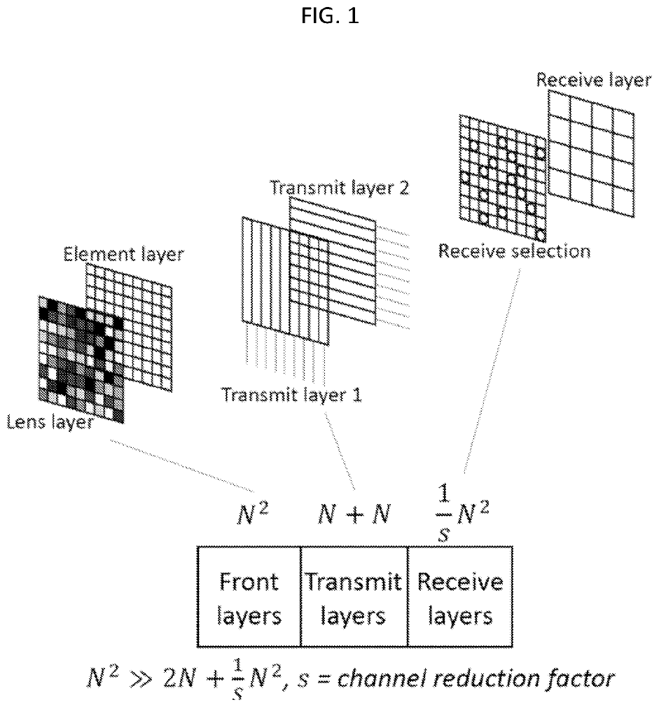

FIG. 1 illustrates an exemplary ultrasound imaging system. As shown in FIG. 1, an element layer (i.e., a collection of transducer elements) can be configured to interact with a lens layer. In some embodiments, the lens layer can be received on the transducer array and/or connected to the common support structure of the ultrasound transducer. The lens layer can be configured to cause a time delay of the transmitted signals from the transmit elements of the transmit layer.

To achieve mechanical delay using an acoustic lens placed in front of an ultrasound transducer array, as shown in FIG. 1, the lens can be configured to introduce a controlled delay to the ultrasound waves. The specific design and properties of the acoustic lens contribute to the mechanical delay effect.

The acoustic lens comprises specific acoustic properties, based on its particular geometry and shape to achieve the desired focusing or steering characteristics. It can have a curved or multi-element structure. Since the speed of sound in the lens material differs from the surrounding medium, this difference in acoustic velocity causes the ultrasound waves to travel at different speeds as they pass through the lens material compared to when they propagate through the surrounding medium. The thickness and curvature of the acoustic lens also contribute to the mechanical delay. The thickness affects the distance the ultrasound waves need to travel through the lens, which introduces a time delay. The curvature of the lens surface can further alter the path length and introduce additional delay. The acoustic lens is configured to focus or steer the ultrasound waves emitted by the transducer elements, and the shape and configuration of the lens causes the waves to be redirected to achieve desired beam characteristics. The mechanical delay introduced by the lens helps shape the transmitted beam, allowing for precise focusing or steering at specific depths or angles.

As shown in FIG. 1, the number of receive elements can be reduced relative to the number of elements of the transducer array, and the number of transmit elements can be further reduced. For example, with N² being the number elements of the transducer array, the relationship can be provided as:

$${N}^{2} ( \# transducer elements ) ≫ 2 N ( \# transmit elements ) + 1 / s {N}^{2} ( \# receive elements ) ,$$

where s=channel reduction factor.

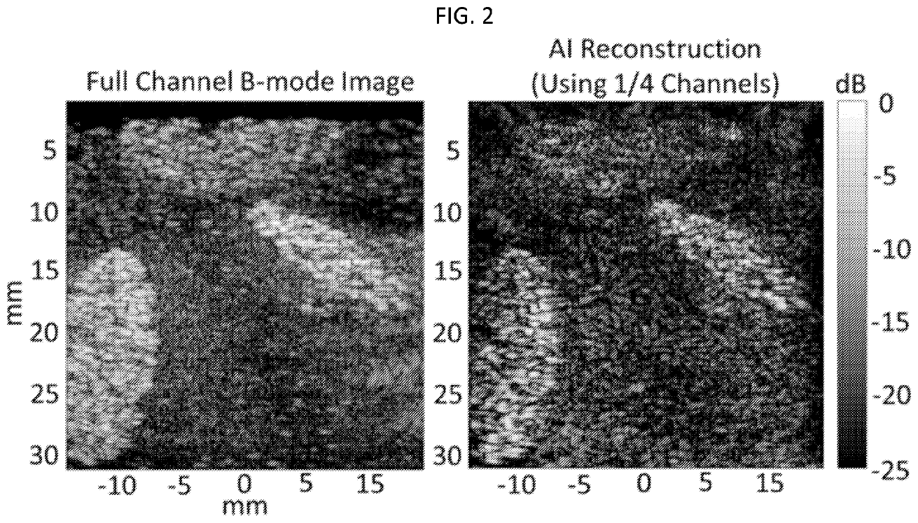

FIGS. 2A and 2B illustrate a full channel B-mode image (FIG. 2A) and an AI reconstruction using ¼ channels (e.g., where s=4).

Sparse arrays can be achieved by activating only a portion of the elements of the transducer array, which effectively reduces the number of electrical channels of the system. In the systems and methods described herein, front-end beamforming can be achieved by a combination of one or more lens layers (e.g., an acoustic lens), which introduces a mechanical delay, and one or more corresponding electrical delay circuits on the selected sub-sampled elements.

The lens layer(s) can comprise a plurality of different lens elements that are positioned over respective ones of transducer elements to affect the signals emitted by the transducer elements. As shown in FIG. 1, these plurality of different lens elements can include one or more groups of lens elements, which create one or more groups of corresponding transducer elements that emit signals in the same time signature. The flexibility of the use of one or more lens layers also allows for significant potential for customization.

In addition, AI-based image formation technology can be used to provide the required beamforming based on the reduced number of receiving sensor elements. Using artificial intelligence (AI) for beamforming image formation in ultrasound can include leveraging machine learning algorithms and computational techniques to enhance the quality and efficiency of the imaging process. In particular, as described herein, the ultrasound system captures ultrasound signals from the transducer elements during the scanning process and the signals contain information about the reflected ultrasound waves from the tissues or structures being imaged. AI can be used to improve beamforming image formation, by providing a collected training dataset paired examples of ultrasound signals and corresponding high-quality beamformed images. Machine learning algorithms can then be are employed to train an AI model using the collected training dataset, with the model learning the underlying patterns and relationships between the raw ultrasound signals and the corresponding beamformed images. The trained AI model can then be applied to the acquired, reduced number of elements, ultrasound signals to predict the beamformed images. The AI-generated beamformed images may undergo postprocessing techniques such as denoising, contrast enhancement, or spatial filtering to further refine the image quality. The processed images can be displayed and visualized for analysis and diagnosis.

The use of AI in beamforming allows for improved image quality, noise reduction, and artifact suppression. It can also optimize the beamforming process to reduce computational complexity and enhance real-time imaging capabilities.

### Example 1

Example 1 illustrates the results of a study performed on a gelatin phantom, where a blood vessel mimicking tube (0.58 mm inner diameter, Polyethylene) was embedded at a depth of 20 mm. To simulate the configuration of a sparse array, a regular 128-channel (element pitch=0.3 mm) L7-4 (5 MHz) transducer was provided with elements intermittently turned off during transmits (Tx) and receives (Rx) to determine its effect on image acquisitions due to the “sparse” configurations.

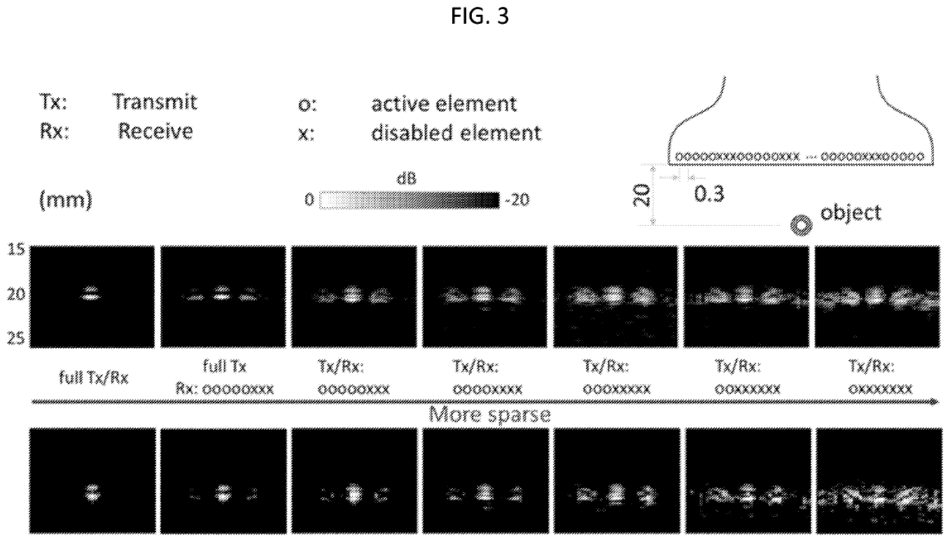

In this example, element #1˜128 were used and all the active elements were fired at the same time. The image field of view is the short axis of the tube. As illustrated in FIG. 3, seven different configurations were tested. In the context of FIG. 3, the “o” denotes an active element while the “x” denotes a disabled element of the ultrasound transducer. The seven different configurations are discussed below, numbered from 1-7 to correspond with the configurations shown from left to right in FIG. 3.
(1) Tx/Rx used all the 128 elements—regular clinical B-mode for best imaging quality;
(2) Tx used all the elements and Rx used elements in a spatially repeated on-off pattern as “00000xxx”;
(3) Tx/Rx used the same on-off pattern as described in (2);
(4)-(7) Incrementally turned off 1 more element in both Tx and Rx until there is only 1 active element out of every 8 consecutive elements.

For each Tx/Rx configurations, the acquired B-mode images used conventional delay-and-sum (DAS) beamforming method (top panel) and delay-multiply-and-sum (DMAS) beamforming (bottom panel). As reflected in the resulting images of FIG. 3, as the elements became more sparse (i.e., moving from left to right) relative to the first configuration (i.e., full Tx/Rx), there is an increased aliasing shown as a “grating lobe” pattern on the image near the expected target signal. One reason is the degraded signal-to-noise ratio (SNR) due to loss of signal components and the interference from grating lobes as the element pitch was enlarged.

As an initiative of overcoming such limitation, the DMAS method can enhance the SNR so that the image of this experiment still meets a certain satisfaction for a sparse configuration of for example, “0000xxxx” (e.g., 1.5 mm between 2 consecutive active elements). However, spatial aliasing can become more pronounced when imaging objects with higher complexity instead of a single tube. In some embodiments, alternative methods which include specific array design and sparse array beamforming may be desirable.

In this example, the vessel phantom was about 1/10th of a typical human femoral artery (e.g., 8-10 mm in diameter). In some embodiments, 16 ultrasound single element transducers (at 5 MHz) with a distance of 1.5 mm can be provided for a sparse array configuration. In some embodiments, 12 elements and an element pitch of 2 mm can be provided with the sparse convolutional beamforming algorithm.

In some embodiments, the channel reduction factor(s) can be 8/5 or greater, 2 or greater, 3 or greater, 4 or greater (e.g., as shown in FIG. 2B) 6 or greater, or 8 or greater. For example, as shown above in the second configuration of FIG. 3, the number of receive layers are reduced, relative to the transmit layers by a channel reduction factor of 8/5. In some embodiments, the channel reduction factor does not exceed 8 (e.g., 8/5≥s≥8). In other embodiments, the channel reduction factor does not exceed 10, 12, or 16.

The transmit channels can be similarly reduced in some embodiments. For example, as shown in the fourth configuration of FIG. 3, the number of active transmit and receive elements are reduced by a factor of 8/5. The reduced number of active transmit and receive elements can be the same (e.g., as shown in the fourth configuration). Alternatively, the number of active transmit elements can be greater than the number of active receive elements.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A sparse-array ultrasound imaging system comprising:
an ultrasound transducer comprising a transducer array having a plurality of transducer elements defining an element layer, the plurality of transducer elements being configured to emit ultrasound signals; and
one or more lens layers positioned over the element layer, the one or more lens layers comprising a plurality of lens elements that correspond, respectively, with the plurality of transducer elements,
wherein the one or more lens layers are configured to affect a time delay of at least some of the signals emitted by the transducer elements.

2. The sparse-array ultrasound imaging system of claim 1, wherein the one or more lens layers comprise a plurality of groups, and lens elements within each group of the plurality of groups are of the same type.

3. The sparse-array ultrasound imaging system of claim 2, wherein different lens layers with the different ones of the plurality of groups have different lens thicknesses.

4. The sparse-array ultrasound imaging system of claim 1, wherein the plurality of groups include at least a first group and a second group, and the one or more lens layers in the first group provide a first time delay of ultrasound signals emitted by corresponding ones of transducer elements, and the one or more lens layers in the second group provide a second time delay to ultrasound signals emitted by corresponding ones of transducer elements, and the first time delay is different from the second time delay.

5. The sparse-array ultrasound imaging system of claim 1, wherein the one or more lens layers are configured to be coupled to the ultrasound transducer of the sparse-array ultrasound imaging system.

6. The sparse-array ultrasound imaging system of claim 5, wherein the one or more lens layers are coupled to the ultrasound transducer at a first spacing relative to the transducer array, and the first spacing is adjustable.

7. The sparse-array ultrasound imaging system of claim 1, wherein a number of receive elements of the transducer array is reduced by a channel reduction factor and wherein the channel reduction factor is 8/5 or greater.

8. The sparse-array ultrasound imaging system of claim 1, wherein a number of receive elements of the transducer array is reduced by a channel reduction factor and wherein the channel reduction factor is 4 or greater.

9. The sparse-array ultrasound imaging system of claim 1, wherein a number of receive elements of the transducer array is reduced by a channel reduction factor and wherein the channel reduction factor is 8 or greater.

10. The sparse-array ultrasound imaging system of claim 7, wherein a number of transmit elements of the transducer array is reduced by the same channel reduction factor as the number of receive elements.

11. A method for ultrasound imaging using a sparse-array ultrasound imaging system, comprising:
providing an ultrasound transducer comprising a transducer array having a plurality of transducer elements defining an element layer, the plurality of transducer elements configured to emit ultrasound signals;
positioning one or more lens layers over the element layer, the one or more lens layers comprising a plurality of lens elements that correspond, respectively, with the plurality of transducer elements, the one or more lens layers being configured to affect a time delay of at least some of the signals emitted by the transducer elements;
emitting ultrasound signals from the transducer elements;
receiving reflected ultrasound signals from a target area by a plurality of active receive elements of the transducer array;
combining the received reflected ultrasound signals affected by the time delay from the one or more lens layers; and
processing the combined signals to generate an ultrasound image of the target area,
wherein a number of the active receive elements of the transducer array is reduced by a channel reduction factor of 8/5 or greater.

12. The method of claim 11, wherein the number of the active receive elements of the transducer array is reduced by a channel reduction factor of 4 or greater.

13. The method of claim 11, wherein the number of the active receive elements of the transducer array is reduced by a channel reduction factor 8 or greater.

14. The method of claim 11, wherein the number of active receive elements of the transducer array is reduced by a channel reduction factor that is less than 16.

15. The method of claim 11, wherein a number of active transmit elements of the transducer array is reduced by the same channel reduction factor as the number of active receive elements.

16. The method of claim 11, wherein the one or more lens layers comprise a plurality of groups, and lens elements within each group of the plurality of groups are of the same type.

17. The method of claim 16, wherein different lens layers with the different ones of the plurality of groups have different lens thicknesses.

18. The method of claim 16, wherein the plurality of groups include at least a first group and a second group, and the one or more lens layers in the first group provide a first time delay of ultrasound signals emitted by corresponding ones of transducer elements, and the one or more lens layers in the second group provide a second time delay to ultrasound signals emitted by corresponding ones of transducer elements, and the first time delay is different from the second time delay.

19. The method of claim 11, wherein the one or more lens layers are configured to be coupled to the ultrasound transducer of the sparse-array ultrasound imaging system.

20. The method of claim 19, wherein the one or more lens layers are coupled to the ultrasound transducer at a first spacing relative to the transducer array, and the first spacing is adjustable.
